(19) United States
(12) Patent Application Publication
(10) Pub. No.: US 2004/0073073 A1
(43) Pub. Date: Apr. 15, 2004

(54) Production of 1,2-dichloroethane

(76) Inventors: Peter Kammerhofer, Burgkirchen (DE); Ingolf Mielke, Burgkirchen (DE); Horst Ertl, Neuoetting (DE); Guenter Staib, Stade (DE)

Correspondence Address: Ronald R Santucci, Frommer Lawrence & Haug, 745 Fifth Avenue, New York, NY, 10151, US

(21) Appl. No.: 10/467,599

(22) PCT Filed: Feb. 13, 2002
(86) PCT No.: PCT/EP02/01503

(30) Foreign Application Priority Data
Feb. 13, 2001 (DE) 10107089.6

(51) Int. Cl. C07C 17/00

(52) U.S. Cl. 570/203

(57) ABSTRACT

A process and an apparatus are described for the preparation of 1,2-dichloroethane by reacting ethene with hydrogen chloride and an oxygen-containing gas in an oxychlorination reactor by means of a fluidised bed with formation of a reaction gas, wherein the reactor gas is filtered outside the oxychlorination reactor by means of at least one filter candle.

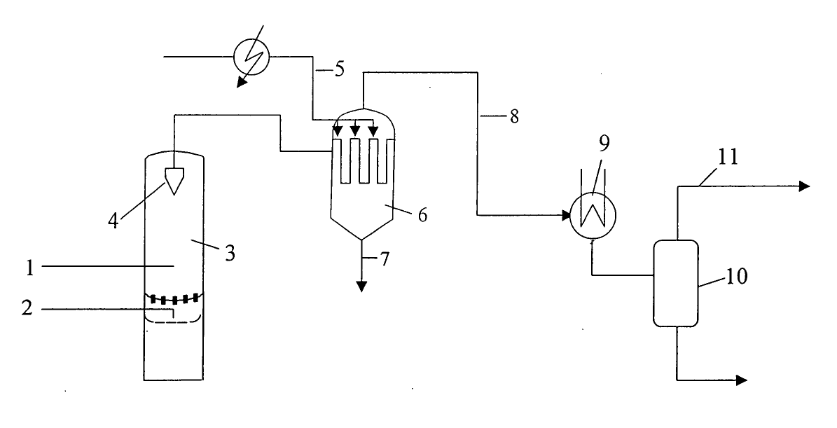

The present invention relates to a process and an apparatus for the preparation of 1,2-dichloroethane by reacting ethene with hydrogen chloride and an oxygen-containing gas in an oxychlorination reactor having a fluidised bed, wherein a reaction gas is formed.

Oxychlorination is understood to be the reaction of an alkene—in this instance ethene—with hydrogen chloride and oxygen or an oxygen-containing gas such as air to form a saturated chlorinated alkane—in this instance 1,2-dichloroethane, also referred to hereinafter as “EDC”-, the reaction taking place according to the equation

C₂H₄+2HCl+1/2O₂⇄Cl—CH₂—CH₂—Cl+H₂0.

The subsidiary reaction product (water) of that reaction can, however, together with the unreacted starting material (hydrogen chloride), form hydrochloric acid, which is very highly corrosive, so that when carrying out such a process appropriately resistant—and, consequently, expensive—materials have to be used for the apparatus in order to carry out the process.

The oxychlorination reaction is carried out in the presence of a catalyst.

In a form of that process which is frequently used on a large industrial scale, a catalyst fluidised bed consisting substantially of copper chloride on an aluminium oxide carrier is used as the catalyst for the oxychlorination reaction.

In the case of that conventional industrial fluidised-bed process, the catalyst is separated out in the upper region of the oxychlorination reactor by a plurality of cyclones arranged one after the other, and consequently the major part is retained in the reactor. In addition, a small portion of the catalyst (the so-called catalyst fragments) passes into the reaction gas leaving the reactor and consequently passes to 1,2-dichloroethane-work-up, where it again has to be separated out.

From German Offenlegungsschrift DE 41 32 030 there is known a process for the removal of catalyst fragments, which are formed in the reaction zone in the preparation of 1,2-dichloroethane by the fluidised-bed oxychlorination process and are passed out from the reaction zone together with the crude EDC gas stream. That removal is carried out in such a way that the catalyst fragments taken away are separated from the crude EDC gas stream in a cleaning zone operated under dry conditions. Preferred embodiments of that process are distinguished by the fact that the catalyst fragments are separated out at a dust separator or in an electrofilter as the cleaning zone, it being possible for the dust separator to be equipped with bag filters which are cleaned with compressed recycle gas. It is then possible for the catalyst fragments separated out in the cleaning zone to be freed from adsorbed reaction products in a desorption zone located downstream.

Such a process avoids the formation of waste water contaminated with heavy metal and inorganic slurry, when the water that is formed and the washing water that is used in working-up are removed. It is, however, disadvantageous that the portion of catalyst separated out, which can no longer be used, has to be discarded and disposed of in proper manner. Furthermore, in the case of such a process, the amount of PCDD/PCDF (polychlorinated dibenzo-p-dioxins/furans) introduced into the environment by way of the waste water from oxychlorination is still considerable. That process also has proved to be very onerous and results in a relatively high apparatus requirement and also, consequently, a high space requirement and high investment costs.

DE-A-197 53 165 discloses a process for the preparation of 1,2-dichloroethane by oxychlorination wherein, in a fluidised bed of a copper-containing catalyst, ethene is reacted with hydrogen chloride and an oxygen-containing gas and the reaction gas emerging from the reactor is freed from catalyst in the reactor by means of very fine filtration and so is retained in the reactor. The reaction gas freed from the catalyst is then passed into a quenching column and condensed in known manner.

A disadvantage of that process is the accumulation of very fine particles in the reactor as a result of the filtration. That is associated with a deterioration in the flow properties of the catalyst bed and with a deterioration in heat transfer in the reactor. Furthermore, the dust loading of the reaction gas is very high because of, inter alia, the absence of the pre-separation by the reactor cyclones. As a result of that high proportion of particles, the filtration surface has to be substantially increased. Furthermore, the process described in

DE 197 53 165 can be applied to existing systems only by onerous means so that that process is used only for new systems and is hardly suitable for existing systems.

It is furthermore known from the prior art (Ullmann's Encyclopedia of Industrial Chemistry, Vol. A6, 1986, p. 269) for the hot reaction gases from the fluidised-bed reactor of oxychlorination, which gases also comprise unreacted HCI gas in addition to EDC and water, to be quenched using an aqueous solution without further treatment. In that method, catalyst fragments that have not been separated out and unreacted hydrogen chloride from the oxychlorination of ethene are washed out. It is possible to use, as washing liquid, both external water and water that is formed during the reaction, the so-called water of reaction. EDC is, together with water from the quench, distilled off and condensed.

In that process it has been found to be disadvantageous that the waste water from the quench has to be disposed of by onerous means because it comprises, amongst other things, the catalyst fragments.

All the known processes of the prior art using fluidised-bed technology have the disadvantage that either only inadequate separation of PCDD/PCDF is possible or, however, an adequate, higher separation rate can be achieved only by onerous means or by complicated process procedures.

A problem of the present invention is therefore to carry out an oxychlorination process for the preparation of 1,2-dichloroethane, using a fluidised bed, in such a manner that the catalyst fragments formed during the reaction can be removed with minimal outlay. Furthermore, it should also be possible therein to achieve as high a degree of separation of catalyst fragments as possible.

It is a further problem of the present invention to achieve adequate separation of PCDD/PCDF from the reaction gas using minimal outlay in terms of apparatus.

The problems are solved in a process of the kind mentioned at the beginning by filtering the reaction gas outside the oxychlorination reactor by means of at least one filter candle.

It has now been found, surprisingly, that filter candles, when filtering the crude 1,2-dichloroethane gas stream under usual conditions, do not present problems either with respect to the ease of carrying out cleaning or with respect to chemical resistance (corrosion resistance). That corrosion resistance was not expected because the filter candles have an extremely large surface area and corrosive gas mixtures flow through them at temperatures of at least 200° C.

In accordance with a preferred embodiment of the present invention, the at least one filter candle comprises, as filter material, at least one ceramic material and/or at least one sintered metal powder and/or at least one wire fleece of high-grade steel (1.4571 or equivalent materials) or another suitable alloy.

For the at least one filter candle, it is possible to use, for example, alloys such as those commercially available under the names ®INCONEL, ®MONEL and/or ®HASTELLOY.

Likewise, filter candles comprising ceramic materials of appropriate average pore sizes can also be used.

When such filter materials are used, especially good separation of PCDD/PCDF from the reaction gas is possible. The separation performance in terms of dioxins and furans is also surprising because material transports of those compounds are very complex and largely unresearched. Especially when the filter candles, or the filter materials used in the filter candles, have an average pore size or separation limit of ≦15 μm, preferably ≦8 μm, more preferably ≦5 μm or ≦1 μm, a dramatic improvement in the separation of PCDD/PCDF can be achieved. As lower limit of the average pore size or separation limit, values of 0.0001 μm, 0.001 μm, 0.01 μm, 0.1 μm and 0.5 μm are entirely possible. Accordingly, the filter materials can have, for example, average pore sizes of from 0.0001 to ≦5 μm. When, on the other hand, fabric filters are used, which have hitherto been regarded as equally effective, no separation of dioxins/furans can be achieved.

When the reaction gases are, in addition, subjected to direct condensation, that is to say condensation without quenching, the small residual amounts of PCDD and/or PCDF that are possibly still present pass virtually entirely into the organic phase, and the waste water from oxychlorination is very largely free from those substances.

Because it is now possible, using the simplest means, to achieve a high separation performance of, especially, polychlorinated dibenzo-p-dioxins/furans (PCDD/PCDF) by using externally located filter elements having at least one filter candle, a drastic reduction in the content of such compounds in the waste water is possible, which waste water otherwise has to be worked up by onerous and expensive means.

It has been possible to solve the problem of very fine particles settling inside the filter housing and, consequently, blocking the filter housing, by means of the following routing of the gas stream in the filter: The speed vector directed in the direction of gravity, resulting from the sinking speed of the catalyst particles and the flow speed of the gas (recycle gas, 1,2-dichloroethane and process water), is greater than 0.

As already described, it is even feasible, in the case of certain applications, for the reaction gas to be condensed directly after very fine filtration, without prior quenching, so that the process step of quenching, which has hitherto always been necessary in the art, can be omitted. As a result, the oxychlorination process is made even simpler and more economical. In the case of such a process, an apparatus can accordingly be used wherein no so-called quenching tower is necessary, resulting in a space saving in the apparatus being used.

The process conditions, especially those of the oxychlorination step by means of a fluidised bed, can be performed preferably in accordance with the process conditions described in the German Auslegeschrift 1 518 931 and the German Patent Specification 1 468 489, the disclosures of which are hereby incorporated by reference in the present description.

Preferably, the reaction gas, after leaving the oxychlorination reactor in a process according to the invention, flows through the filter candle at a pressure of about from 1 to 6 bar, preferably of about 3.5 bar.

In the process, the reaction gas should have a temperature of about from 200 to 250° C., preferably about 220° C., on flowing through the filter.

It has been possible to obtain especially good results in a process according to the invention, when the speed vector directed in the direction of gravity, resulting from the sinking speed of the catalyst particles and the flow speed of the gas (recycle gas, 1,2-dichloroethane and process water), is greater than 0, especially greater than 10 mm/second.

After filtering, the reaction gas could, depending upon the degree of purification, be passed into a quenching column or directly condensed.

In such a process, it is also possible to use a very simply constructed apparatus because the catalyst fragments do not have to be removed using other complicated apparatuses such as those described, for example, in DE 197 53 165.

Preferably, the content of polychlorinated dibenzo-p-dioxins/furans (PCDD/PCDF) in the 1,2-dichloroethane after a process according to the invention is less than 0.1 μg/to of 1,2-dichloroethane.

The degree of separation of the entrained catalyst particles in the oxychlorination process by means of a fluidised bed is, in the case of a process of the invention according to a preferred embodiment, more than 99.99%.

As a result of the fact that the filtration is carried out using a filter candle used for carrying out a process according to the invention, which is located outside the oxychlorination reactor, retro-fitting of existing oxychlorination systems having a fluidised bed is possible in very simple manner. That is especially very simply possible because the filters used having at least one filter candle can be constructed very compactly.

Because of the high operational reliability of the filter candles it is also possible, moreover, to omit a back-up unit.

For the reason, inter alia, that the filter candles are subject to practically no wear, they have an unlimited life, especially as, in the case of the stream routing according to the invention in the filter candle, the particles filtered out can be readily removed. That has also proved to be advantageous because no onerous maintenance work has to be performed.

Especially in order to be able to carry out a process according to the invention especially well, the apparatus for the preparation of 1,2-dichloroethane according to the oxychlorination process in a fluidised-bed reactor should have inlets for hydrogen chloride and oxygen-containing gas that lead directly into the fluidised bed of the oxychlorination reactor.

Those inlets may comprise porous, gas-permeable packing elements.

It is also especially advantageous when the ethene and the recycle gas stream are passed into the oxychlorination reactor through a tray made from porous, gas-permeable material or provided with packing elements of porous, gas-permeable material, which assists the formation of the fluidised bed.

The construction of the inlets could be selected, for example, in the form described in DE 199 03 335 A1, which is hereby to be incorporated by reference in the description.

Further advantages and developments of the invention are shown by the patent claims, the drawing, and the following description in which exemplary embodiments of the invention are described in detail with reference to the drawing.

The single FIGURE shows, by way of example, an apparatus according to the invention in accordance with a preferred embodiment.

The process according to the invention is also illustrated hereinbelow with reference to the drawing.

The pre-heated starting materials, hydrogen chloride and oxygen, are passed, by way of a pipe 1, directly into a reaction space of a reactor 3. At the same time, by way of a further pipe 2, the pre-heated recycle gas and ethene are passed into the reactor 3. In the reactor 3 there is a catalyst (not shown in the Figure). The crude 1,2-dichloroethane, the process water and the recycle gas are passed, by way of cyclones 4, into the very fine filter 6. The separated very fine catalyst dust is removed from the system by way of an outlet 7. By way of a third pipe 5, the pre-heated back-washing gas for cleaning (back-washing) the filter elements is passed into the filter 6. The crude 1,2-dichloroethane, the process water and the recycle gas without a significant content of very fine particles are passed, by way of a further pipe 8, into a condenser 9, wherein the EDC and the water vapour are condensed. The liquid EDC/water mixture is separated from the recycle gas in a separator 10 and sent, by way of a pipe 12, for working-up. The recycle gas is returned, by way of a pipe 11, to the inlet side of the recycle gas compressor.

### EXAMPLE 1

An oxychlorination reactor having a fluidised bed is used for the preparation of 1,2-dichloroethane, with CuCl₂ being used as catalyst. The oxychlorination is carried out therein under conventional process conditions, which will be known to the person skilled in the art and are therefore not explicitly mentioned.

After leaving the fluidised-bed reactor, the reaction gas (500 Nm³/h) flows through a very fine filter of a cleaning zone, operated under dry conditions, at a temperature of from 200 to 250° C. (preferably 220° C.) and at a pressure of about from 1 to 6 bar (preferably 3.5 bar) for separating out entrained catalyst particles, the so-called catalyst fragments, as a result of which the catalyst is practically completely separated out.

The very fine filter consists of 8 filter elements. The filter elements are made from the material 316 S (stainless steel). The 8 filter elements are periodically back-washed by means of automatic valves. Pre-heated nitrogen, which is made available at a pressure of 8 bar, is used as back-washing gas. The very fine dust separated out in the filter cone is removed once per day by way of an outlet.

The routing of the reaction gas therein is so provided that the speed vector directed in the direction of gravity, resulting from the sinking speed of the catalyst particles and the flow speed of the gas (recycle gas, 1,2-dichloroethane and process water), is greater than 0.

As a result, optimum settling of the very fine particles is ensured.

The reaction gas freed from catalyst fragments, having a temperature of from 200 to 250° C., preferably about 220° C., is then passed through a line into a condenser, where the EDC and the production water are condensed. The condensed liquid is separated from the recycle gas in a gas separator. The EDC/water mixture is passed, by way of a pipe, to a separating container, in which the aqueous phase is separated from the EDC.

The following concentrations of polychlorinated dibenzo-p-dioxins/furans were measured in the waste water from oxychlorination:

| | | | sintered metal filter |
|---|---|---|---|
| | | | (stainless steel) |
| | without | fabric | having an average |
| | filter | filter | pore size < 5 μm |
| PCDD/PCDF TEQ according to | 5 | 4.5 | <0.1 |
| NATO/CCMS in μg/t of OC | | | |
| capacity | | | |

Explanation of abbreviations:
PCDD = polychlorinated dibenzodioxins
PCDF = polychlorinated dibenzofurans
TEQ = toxicity equivalent
NATO/CCMS = North Atlantic Treaty Organization/Committee on the Challenges of Modern Society
μg = microgram
t of OC = tonne of oxychlorination capacity

That means that, using a process according to the invention, it is possible, using filter candles having a specific average pore size, for the PCDD/PCDF content after filtration to be reduced by more than 97% compared to filtration using fabric filters customarily used in the art.

The degree of separation of entrained catalyst particles is accordingly more than 99.99%. The content of polychlorinated dibenzo-p-dioxins/furans in the waste water can therefore, as a consequence thereof, be dramatically reduced. There is accordingly no longer a need to carry out high-cost and time-consuming disposal of the waste water after oxychlorination.

1. Process for the preparation of 1,2-dichloroethane by reacting ethene with hydrogen chloride and an oxygen-containing gas in an oxychlorination reactor with formation of a reaction gas, characterised in that the reaction gas is filtered outside the oxychlorination reactor by means of at least one filter candle.

2. Process according to claim 1, characterised in that the filter material used for the at least one filter candle comprises ceramic materials, sintered metal powders, wire fleece of high-grade steel or high-grade steel alloys.

3. Process according to one of the preceding claims, characterised in that the filter material used for the at least one filter candle has an average pore size of ≦15 μm, preferably ≦8 μm and more preferably ≦5 μm.

4. Process according to one of the preceding claims, characterised in that the reaction gas flows through the filter candle at a pressure of about from 1 to 6 bar.

5. Process according to one of the preceding claims, characterised in that the reaction gas flows through the filter candle at a temperature of about from 200 to 250° C.

6. Process according to one of the preceding claims, characterised in that the routing of the gas stream in the filter candle is so selected that the speed vector directed in the direction of gravity, resulting from the sinking speed of the particles being filtered out, such as the catalyst particles, and the flow speed of the gas (recycle gas, 1,2-dichloroethane and process water), is greater than 0.

7. Process according to one of the preceding claims, characterised in that, after filtering, the reaction gas is passed into a quenching column or is directly condensed.

8. Process according to one of the preceding claims, characterised in that the content of polychlorinated dibenzo-p-dioxins/furans (PCDD/PCDF) is less than 0.1 /μg/t of 1,2-dichloroethane in the waste water from fluidised-bed oxychlorination.

9. Process according to one of the preceding claims, characterised in that the degree of separation of catalyst fragments from the reaction gas is more than 99.99%.

10. Apparatus for the preparation of 1,2-dichloroethane by reacting ethene with hydrogen chloride and an oxygen-containing gas, especially for use in a process according to one of the preceding claims, which has an oxychlorination reactor and a filter, characterised in that the filter has at least one filter candle, which is arranged outside the oxychlorination reactor.

11. Apparatus according to claim 10, characterised in that the at least one filter candle comprises ceramic materials, sintered metal powders, wire fleeces of high-grade steel or high-grade steel alloys.

12. Apparatus according to claim 10 or 11, characterised in that the filter material used for the at least one filter candle comprises ®INCONEL, ®MONEL and/or ®HASTELLOY.

13. Apparatus according to one of claims 10-12, characterised in that the filter material used for the filter candle, of which there is at least one, has an average pore size of from 0.01 to 15 μm, preferably from 0.1 to 8 μm, more preferably from 0.2 to 5 μm, and most preferably from 0.8 to 3 μm.

14. Apparatus according to one of claims 10 to 13, characterised in that the inlets for hydrogen chloride and/or the oxygen-containing gas pass directly into the fluidised bed of a fluidised-bed reactor.

15. Apparatus according to claim 14, characterised in that the inlets comprise porous, gas-permeable packing elements.

16. Apparatus according to one of claims 10 to 15, characterised in that the recycle gas and/or the ethene is passed into the oxychlorination reactor through a tray of porous, gas-permeable material.
